(19) United States
(12) Patent Application Publication
(10) Pub. No.: US 2023/0127903 A1
(43) Pub. Date: Apr. 27, 2023

(54) AIML TO MONITOR CLINICAL PROTOCOL DEVIATIONS

(71) Applicant: IQVIA Inc., Danbury, CT (US)

(72) Inventors: Hao Ran Laurence Lin, Apex, NC (US); Jonas Renstroem, Uppsala (SE)

(21) Appl. No.: 17/512,205

(22) Filed: Oct. 27, 2021

(51) Int. Cl. G16H 50/70; G16H 10/20; G16H 10/60; G16H 40/20; G06N 20/00

(52) U.S. Cl. CPC G16H 50/70; G16H 10/20; G16H 10/60; G16H 40/20; G06N 20/00

(57) ABSTRACT

A method includes patient data from a centralized database to identify protocol deviations from the patient data. Natural language processing or machine-learning is performed by a cloud computing server to perform content extraction on the protocol deviations, wherein the content extraction is performed to extract keywords, phrases, and supervised text, wherein the extracted keywords, phrases, and supervised text are used to group the protocol deviations by content. The method also includes reporting, to a user interface, multiple statistical summaries of the protocol deviations, wherein the multiple statistical summaries include a patient, site, study, and country.

### TECHNICAL FIELD

The present disclosure generally relates to monitor protocol deviations of patient data from a centralized database system.

### BACKGROUND

Protocol deviations are typically unplanned or unforeseen at clinical trials at any stage. A protocol deviation is a short text documenting the unplanned excursion and is carefully reviewed by site-specific clinical staffs to follow up on patients for their safety.

Current existing solutions for identifying or locating protocol deviations include spreadsheets of protocol deviations. Other current solutions including manual keyword search and manual counting of the protocol deviations.

Current outcomes of protocol deviations include studying the level of protocol deviations for each month of the clinical trials. Other outcomes include indirect information on the protocol deviations.

Current outcomes also have no good solution. As such, there is a basic summation of protocol deviations from every patient and a manually look-up of the patient IDS and site IDS. In addition, other problems include no detailed patient profile that is generated. In other words, the profile has to be manually copied and pasted from multiple datasets with spreadsheets.

Accordingly, a need exits for a more efficient means of identifying the protocol deviations in the clinical data, and statistically summarizing the protocol deviations while having an efficient means of generating a detailed chronological profile for each patient and clinical study site.

### SUMMARY

The following summary is provided to facilitate an understanding of some of the features of the disclosed embodiments and is not intended to be a full description. A full appreciation of the various aspects of the embodiments disclosed herein can be gained by taking the specification, claims, drawings, and abstract as a whole.

The aforementioned aspects and other objectives can now be achieved as described herein.

In an embodiment, a method includes collecting a set of patient data from a centralized database, wherein the patient data is collected to identify protocol deviations within the patient data. The method also includes performing natural language processing and/or machine-learning by a cloud-computing server on the protocol deviation to perform content extraction on the protocol deviations, wherein the content extraction is performed to extract keywords, phrases, and supervised text. The extracted keywords, phrases, and supervised text are used to group the protocol deviations by content. The method also includes reporting, to a user interface, multiple statistical summaries of the protocol deviations, wherein the multiple statistical summaries include a patient, site, study, and country.

A summary of analytical results of the protocol deviations are reported to the user interface.

A chronological view for each patient and site identifier is provided to the user interface.

The method includes identifying protocol deviation trends and along with patient visits among the patient data received from the centralized database.

In an embodiment, a method includes pulling datasets to patient identifiers and clinical operations from a database system. The method also includes performing natural language processing of the pulled datasets related to the patient identifiers and clinical operations by a processing unit within a cloud-computing server. The natural language processing and other statistical/AIML bases analytics are performed to identify trends and correlations among protocol deviations found within the datasets including patient visits, and classify and group the protocol deviations within the datasets. The protocol deviation groups are obtained by keywords, phrases, and supervised text that are extracted by the natural language processing. The method also includes storing analytical results of the protocol deviations from the natural language processing and the other statistical/AIML base analytics in an analytical management system.

The processing unit continuously receives updated patient and protocol deviation data from the database system for all on-going studies.

The processing unit uses artificial intelligence and machine-learning to extract the keywords, phrases, and supervised texts from the datasets.

The method also includes making copies of the protocol deviations with corrected spelling and standardized names.

In an embodiment, a system includes a central database system with a set of non-identifiable patient data and study site information, wherein the patient data, site level data, and study site information include protocol deviations. The system also includes a cloud computing server that pulls the set of patient data from the central database system and performs natural language processing and/or machine-learning on the protocol deviations to perform content extraction on the protocol deviations. The content extraction is performed to extract keywords, phrases, and supervised text. The extracted keywords, phrases, and supervised text are used to group the protocol deviations by content. The system also includes a user interface receiving multiple statistical summaries of the protocol deviations from the cloud-computing server. The multiple statistical summaries include a patient site, study, and country.

The cloud-computing server reports a dynamic content dashboard to the user interface that requires authorized access per study and prevents identifiable study or patient information shared between studies.

Problems are identified from the protocol deviations.

Each patient identifier per each visit is statistically summarized.

### BRIEF DESCRIPTION OF THE FIGURES

The accompanying figures, in which like reference numerals refer to identical or functionally similar elements throughout the separate views and which are incorporated in and form a part of the specification, further illustrate the present invention and, together with the detailed description of the invention, serve to explain the principles of the present invention.

FIG. 1 illustrates a monitoring system in accordance with an embodiment of the invention;

FIG. 2 illustrates a graph of protocol deviation trends in accordance with the invention;

FIGS. 3(a) and 3(b) illustrate other graphs with statistical summaries of patient visits in accordance with an embodiment of the invention; and

FIG. 4 energy illustrates a comparison graph with other studies of protocol deviations according to an embodiment of the invention.

FIG. 5 is a flowchart in accordance with an embodiment of the invention.

FIG. 6 depicts a system in accordance with an embodiment of the invention

Unless otherwise indicated illustrations in the figures are not necessarily drawn to scale.

### DETAILED DESCRIPTION OF SOME EMBODIMENTS

### Background and Context

The particular values and configurations discussed in these non-limiting examples can be varied and are cited merely to illustrate one or more embodiments and are not intended to limit the scope thereof.

Subject matter will now be described more fully herein after with reference to the accompanying drawings, which form a part hereof, and which show, by way of illustration, specific example embodiments. Subject matter may, however, be embodied in a variety of different form and, therefore, covered or claimed subject matter is intended to be construed as not being limited to any example embodiments set forth herein, example embodiments are provided merely to be illustrative. Likewise, a reasonably broad scope for claimed or covered subject matter is intended. Among other issues, subject matter may be embodied as methods, devices, components, or systems. The followed detailed description is, therefore, not intended to be interpreted in a limiting sense.

Throughout the specification and claims, terms may have nuanced meanings suggested or implied in context beyond an explicitly stated meaning. Likewise, phrases such as “in one embodiment” or “in an example embodiment” and variations thereof as utilized herein may not necessarily refer to the same embodiment and the phrase “in another embodiment” or “in another example embodiment” and variations thereof as utilized herein may or may not necessarily refer to a different embodiment. It is intended, for example, that claimed subject matter include combinations of example embodiments in whole or in part.

In general, terminology may be understood, at least in part, from usage in context. For example, terms such as “and,” “or,” or “and/or” as used herein may include a variety of meanings that may depend, at least in part, upon the context in which such terms are used. Generally, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures, or characteristics in a plural sense. Similarly, terms such as a “a,” “an,” or “the”, again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

One having ordinary skill in the relevant art will readily recognize the subject matter disclosed herein can be practiced without one or more of the specific details or with other methods. In other instances, well-known structures or operations are not shown in detail to avoid obscuring certain aspects. This disclosure is not limited by the illustrated ordering of acts or events, as some acts may occur in different orders and/or concurrently with other acts or events. Furthermore, not all illustrated acts or events are required to implement a methodology in accordance with the embodiments disclosed herein.

Unless defined otherwise, all technical and scientific terms used herein have the same meanings as commonly understood by one of ordinary skill in the art to which the disclosed embodiments belong. Preferred methods, techniques, devices, and materials are described, although any methods, techniques, devices, or materials similar or equivalent to those described herein may be used in the practice or testing of the present invention.

Although claims have been included in this application to specific enumerated combinations of features, it should be understood the scope of the present disclosure also includes any novel feature or any novel combination of features disclosed herein.

References “an embodiment,” “example embodiment,” “various embodiments,” “some embodiments,” etc., may indicate that the embodiment(s) so described may include a particular feature, structure, or characteristic, but not every possible embodiment necessarily includes that particular feature, structure, or characteristic.

Headings provided are for convenience and are not to be taken as limiting the present disclosure in any way.

Each term utilized herein is to be given its broadest interpretation given the context in which that term is utilized.

### Terminology

The following paragraphs provide context for terms found in the present disclosure (including the claims):

The term “patient” regards to the participants in a clinical trial. Once enrolled, patients are assigned to unique study IDs. Any traceable information (e.g., phone number, address, I.P. address, driving license, . . . etc) that can reveal the patient real-life identity are not accessible by the disclosed embodiments.

The term “patient data” refers to patient health and vital information that are generated throughout the study. Patient data may include
Patient vital signs and lab sample results
Patient medical history
Patient study ID (not traceable to patient real-life identity from the centralized clinical trial monitoring team)
Patient visit schedule
Patient protocol deviation documents
Basic Patient demographic information (e.g., age, gender, race, ethnicity, country)

The term “Site” refers to a clinical trial investigational site where clinical trial participates health/vital data are collected regularly and protocol deviations (PDs) are identified and documented. The transitional term “comprising”, which is synonymous with “including,” “containing,” or “characterized by,” is inclusive or open-ended and does not exclude additional, unrecited elements or method steps. See, e.g., Mars Inc. v. H. J. Heinz Co., 377 F.3d 1369, 1376, 71 USPQ2d 1837, 1843 (Fed. Cir. 2004) (“[L]ike the term ‘comprising,’ the terms ‘containing’ and ‘mixture’ are open-ended.”). “Configured to” or “operable for” is used to connote structure by indicating that the mechanisms/units/components include structure that performs the task or tasks during operation. “Configured to” may include adapting a manufacturing process to fabricate components that are adapted to implement or perform one or more tasks.

“Based On.” As used herein, this term is used to describe factors that affect a determination without otherwise precluding other or additional factors that may affect that determination. More particularly, such a determination may be solely “based on” those factors or based, at least in part, on those factors.

All terms of example language (e.g., including, without limitation, “such as”, “like”, “for example”, “for instance”, “similar to”, etc.) are not exclusive of other examples and therefore mean “by way of example, and not limitation . . . ”.

A description of an embodiment having components in communication with each other does not infer that all enumerated components are needed.

A commercial implementation in accordance with the scope and spirit of the present disclosure may be configured according to the needs of the particular application, whereby any function of the teachings related to any described embodiment of the present invention may be suitably changed by those skilled in the art.

The flowchart and block diagrams in the figures illustrate the architecture, functionality, and operation of possible implementations of systems and methods according to various embodiments. Functions noted in the block may occur out of the order noted in the figures. For example, two blocks shown in succession may, in fact, be executed substantially concurrently, or the blocks may sometimes be executed in the reverse order, depending upon the functionality involved.

Further, any sequence of steps that may be described does not necessarily indicate a condition that the steps be performed in that order. Some steps may be performed simultaneously.

The functionality and/or the features of a particular component may be alternatively embodied by one or more other devices that are not explicitly described as having such functionality/features. Also, various embodiments of the present invention need not include a device itself.

More specifically, as will be appreciated by one skilled in the art, aspects of the present invention may be embodied as a system and/or method. Furthermore, aspects of the present invention may take the form of a plurality of systems to enable gas meter to perform self-checking to determine its overall functioning without requiring a meter operator.

### Introduction

Embodiments of the present invention include a system for pulling large datasets of patient data from a centralized database system. The datasets from the centralized database system will include PD data. A cloud-computing server within a cloud-commutating platform will pull the large datasets from the centralized data system. The datasets will include patient/lab data, site visit/activity data, and PD data.

Once the cloud-computing server has pulled the data, natural language processing can be done to extract content from the PDs. Keywords, phrases, and semi-supervised or supervised text is extracted from the PDs to find any trends or statistical summaries of the PDs.

The trends and correlations of the PDs can be identified. In particular, for each patient, it can be identified where the PDs are more likely to occur. The PDs can be more likely to occur with patients at the second patient visit, but not as likely at screening or at a later patient visit. Moreover, the PDs can have the same monthly trends.

As a result of highlighting the trends of the PDs, the PDs per patient visits can be summarized. Sites and patients who show inconsistent and problematic behavior can be identified. Moreover, there can be a detailed and chronological view for each patient per patient visit.

The PDs of the current dataset can also be compared to the PDs of historical or baseline studies to see if there are any similarities or differences between the current dataset and the baseline studies. A better understanding of the current PDs can lead to better statistical summaries based on the comparison with historical/baseline studies.

Accordingly, multiple visual options will be able to summarize analytical results. Moreover, there will be multiple statistical summaries of the PDs at various levels. The various levels of the PDs include the site, the type of duty, the country, month, and also even the quarter within the calendar year. In addition, there will be a detailed chronological view on each participant/patient and each site.

### System Structure

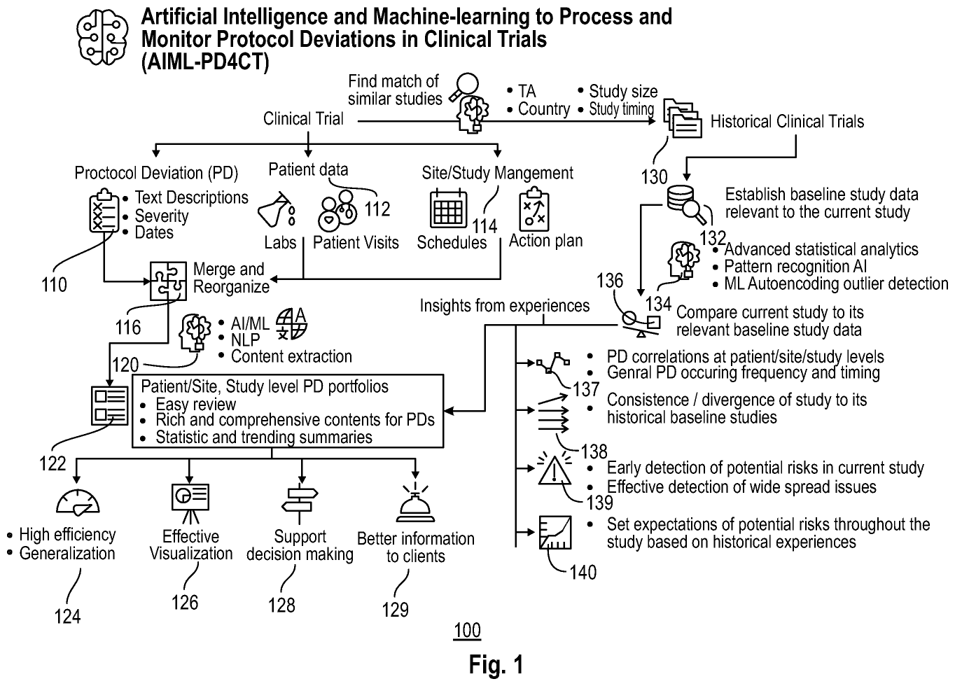

FIG. 1 illustrates a monitoring system 100. Patient data or datasets are pulled from a centralized database system. A cloud-computing server from a cloud-commutating platform will pull the datasets from the centralized database system. The received data will have PDs. The cloud-computing server configured. will identify the PDs 110 within the received data. The PDs 110 will include text descriptions, the severity, and the dates involving the patient data. The patient data 112 will include all of the patient visits and the labs involved. The data. will also include site/study management 114. The site/study management 114 will include the patient schedules and the action plans that are involved. The cloud-computing server will also merge and reorganize 116 the PDs 110. Once the PDs 110 are merged and reorganized 116, the cloud-computing server will perform artificial intelligence or machine-learning 120 on the PDs 110. The cloud-computing server will also perform natural language processing and content extraction 120.

In FIG. 1, the cloud-computing server performs the content extraction 120 of the PDs 110 to identify keywords and important phrases in the PDs 110. In addition, the context extraction 120 involves extracting semi-supervised or supervised artificial intelligence and machine-learning text. The content is extracted to group the PDs 110 by their content. The cloud-computing server will provide a patient/site study level 122 of the PDs 110. The patient/site study level 122 will include a review of the PDs 110. Further, the patient/site study level 122 will include comprehensive contents for the PDs 110 and also statistic and trending summaries. As such, any trends or correlations of the PDs 110 will be identified. The cloud-computing server will also provide high efficiency generalization 124, effective visualization 126, decision-making support 128, and better information to clients 129.

Still Referring to FIG. 1, the cloud-computing server can compare the PDs 110 with historical studies/historical clinical trials 130. In other words, the cloud-computing server can find similar studies of other PDs or match the PDs 110 of the current patient data with PDs of other datasets. The historical studies 130 can also include study size, study timing, country and/or time (TA). The cloud-computing server will also establish study data 132. The study data 132 will be relative to the current patient data. The cloud-computing server can also perform advanced statistical analysis 134 that includes pattern recognition artificial intelligence and machine-learning auto-encoding outlier detection of the PDs 110.

In FIG. 1, the cloud-computing server can perform a comparison 136 of the current study of the PDs 110 to its relative baseline studies. In other words, the PDs 110 can be compared to similar studies that include other PDs by the comparison 136, wherein the current study is compared to relevant baseline study data. Further, the PD correlations 137 are various levels are obtained. The PD correlations 137 will be at the patient level, site level, and study level that include general PDs relative to their frequency and timing. Moreover, the cloud-computing server can also include a consistence/divergence 138 of study to its historical baseline studies. The consistence/divergence 138 of the PDs 110 can be compared to historical baseline studies with other PDs. The cloud-computing server can identify how the current PDs 110 compare to the historical baseline studies. The cloud-computing server can also provide early detection 1.39 for potential risks in the current study of the PDs 110. The early detection 139 can include effective detection of widespread issues. The cloud-computing server can also set expectations 140 for potential risks that can occur throughout the study based on the historical experiences of previous studies with PDs.

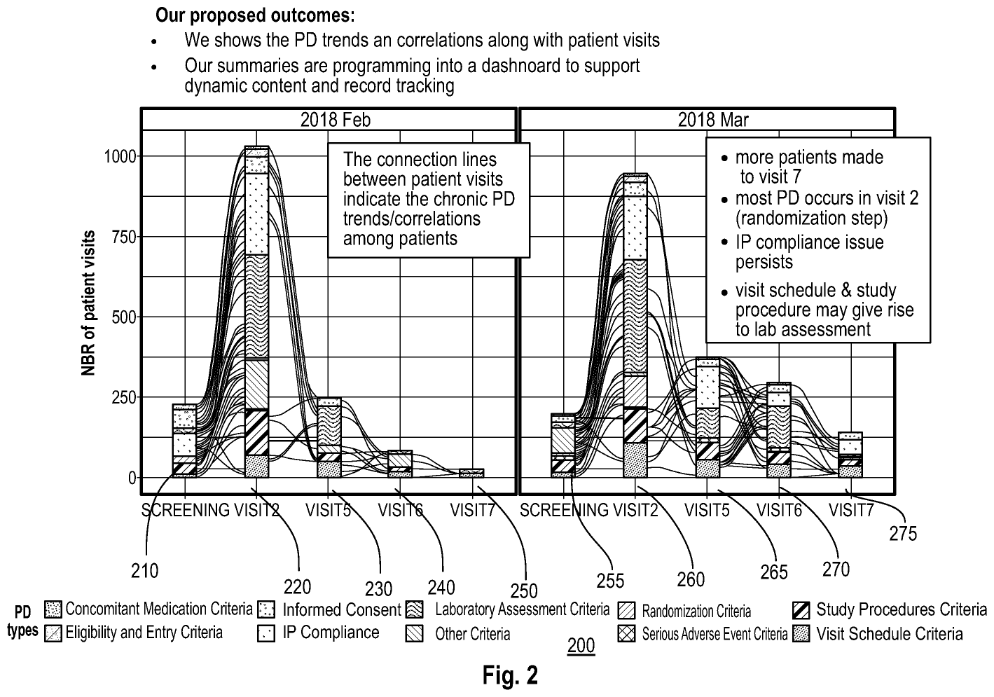

In FIG. 2, a graph 200 of proposed outcomes is shown. The PD trends and correlations along with patient visits are illustrated. In addition, the summaries of the PDs are programmed into a dashboard to support dynamic content and record tracking. The graph 200 illustrates screening 210, visit2 220, visit5 230, visit6 240, and visit7 250 for the first quarter Q1 of 2018. The graph 200 also illustrates screening 255, visit2 260, visit5 265, visit6 270, and visit7 275 for the second quarter Q2 of 2018. The screening 210, 255, visit2 220, 260, visit5 230, 265, visit6 240, 270 and visit7 250, 275 include various PD types. The PD types include concomitant medication criteria, eligibility and entry criteria, informed consent, IP compliance, laboratory assessment criteria, other criteria, randomization criteria, serious adverse event criteria, study procedures criteria, and visit schedule criteria.

In FIG. 2, from the graph 200, the trends of the PD types can be identified. For instance at vi.sit2 220, 260, there is a higher incident of the PD types both in the Q1 and Q2 periods of 2018. PD types such as IP compliance and Laboratory visits had greater incidents at visit2 220, 260 for both the first and second quarters Q1, Q2 of 2018. In addition, the other PD types such as study procedures, visit schedule criteria and randomization criteria have a greater occurrence at visit2 220, 260. In contrast, at visit6 240, 270 and visit7 250, 275, the PD types are not as prevalent. As such, the various illustrations on the graph 200 show when the PD types are more likely to occur and when the PD types are not as likely to occur. The PD trends appear consistently from screening 210, 255 to visit7 250, 275 for both Q1 and Q2 of 2018.

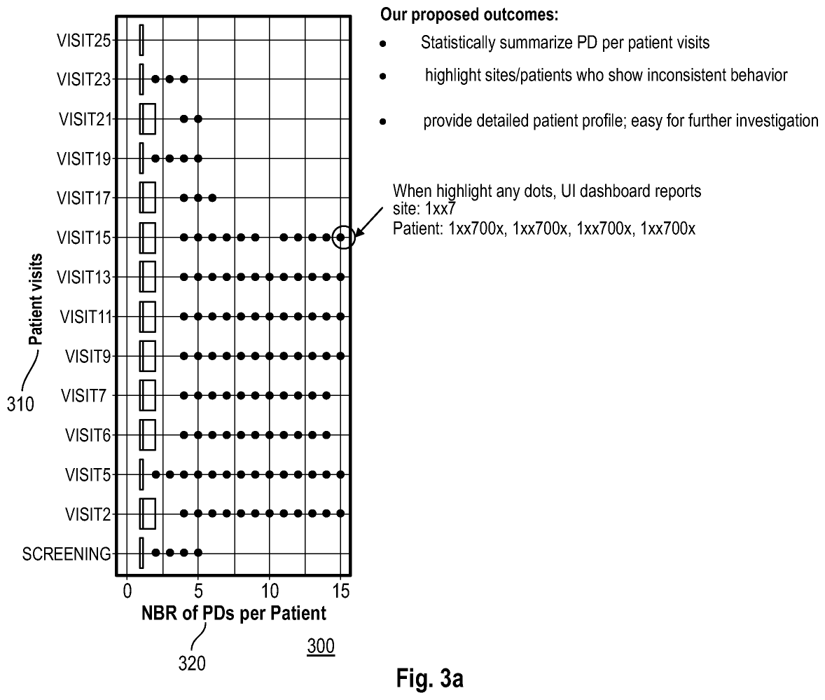

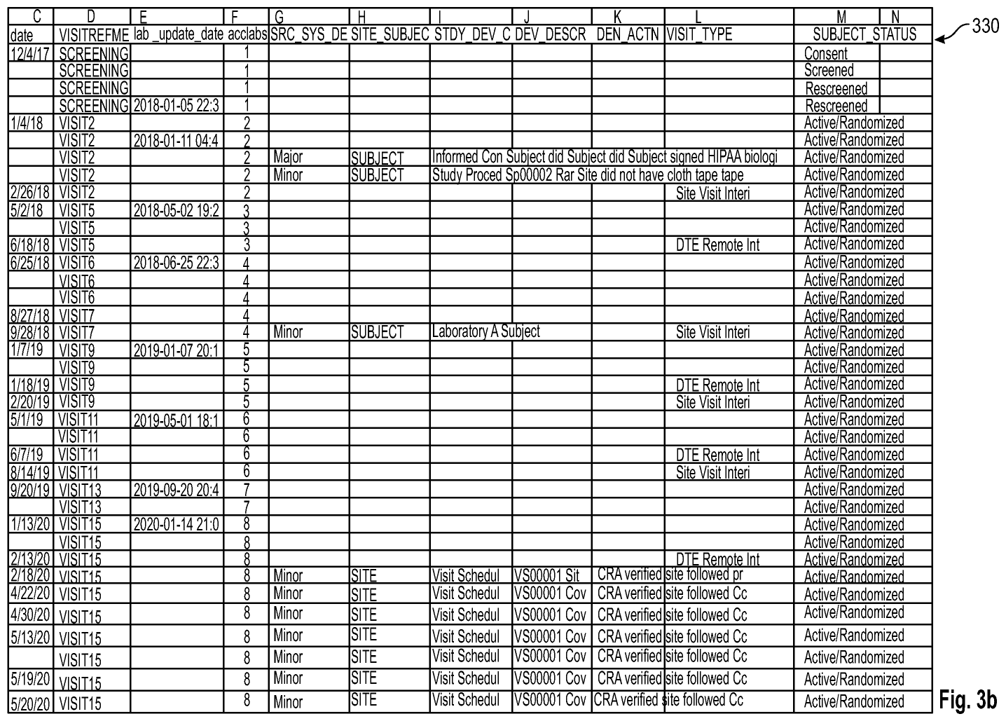

Referring to FIGS. 3(a) and 3(b), graphs 300 and 330 of proposed outcomes are shown. A trend of where PDs are more likely to occur per patient is identified based on graphs 300 and 330 of the proposed outcomes. The graph 300 includes patient visits 310. The patient visits include a patient screening followed by the number of visits. The graph 300 also includes the number (NBR) of PDs per visit 320. As can be seen from the graph 330 of proposed outcomes, the amount of PDs increase from Visit2 to Visit15, but are not as prevalent at Screening or after Visit15. As such, a trend or correlation is identified by identifying when the PDs are more likely to occur per patient. In addition, the patient sites and patients that show inconsistent behavior are highlighted. Further, a detailed patient profile for each patient can be made based on the data from the screening and each of the visits relative to the patient visits 310 and :PDs per patient 320.

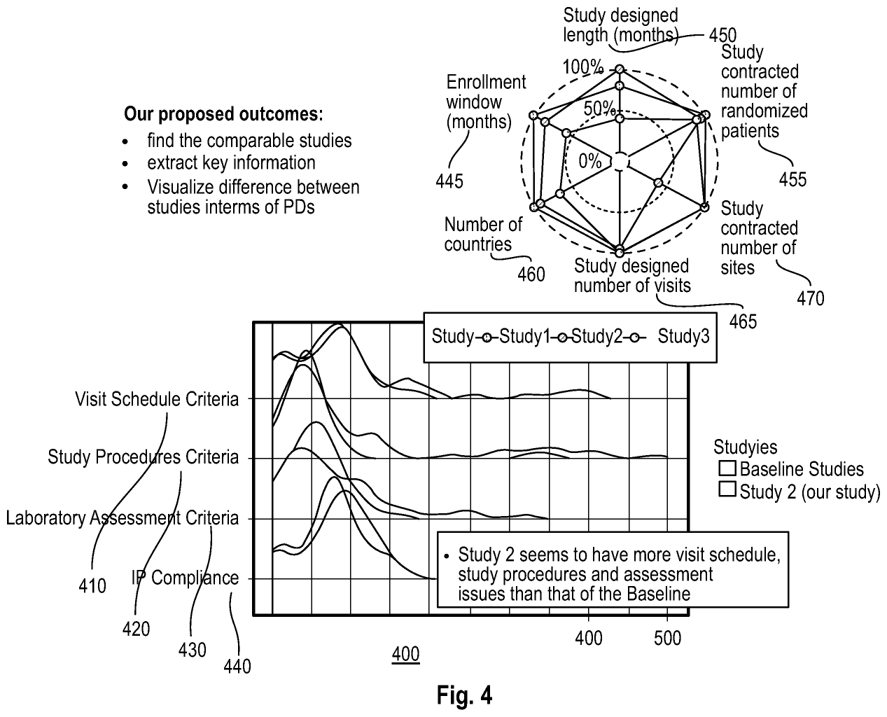

Referring to FIG. 4, a graph 400 illustrating a comparison of the PDs from the database of the centralized database with PDs from other studies is illustrated. The factors shown in the graph 400 include visit schedule criteria 410, study procedures criteria 420, laboratory assessment criteria 430, and IP compliance 440. Study 2 is compared to other baseline studies with regard to visit schedule criteria 410, study procedures criteria 420, laboratory assessment criteria 430, and ip compliance 440. From the graph 400, study 2 has more visit schedule, study procedures, and lab assessment issues than the baseline studies. In other words, there appear to be more PDs in study 2 than the baseline studies. The purpose of comparison is to find comparable studies, extract key information, and visualize the difference between the studies in terms of PDs.

In FIG. 4, levels of the PDs relative to number of countries 460, study designed number of visits 465, study contracted number of sites 470, study contracted number of randomized patients 455, study designed length 450, and enrollment windows 445 are illustrated between different studies Study 1, Study 2, and Study 3. A comparison of Study 1, Study 2, and Study 3 using the different PD levels from number of countries 460 to enrollment windows 445 show how the various PD levels compare among Study 1, Study 2, and Study 3. Moreover, with regard to study contracted number of sites 470 and study designed number of visits 465, the PD levels appear similar. With respect to enrollment windows 445 and study designed length 450, the PD levels appear to be different. Nevertheless, by comparing the PD levels among the various studies, they can identify certain trends among the studies and certain differences of the various studies and have a better statistical summary of the data obtained from the centralized database. Moreover, not all PDs are equal. Further statistical investigations can also be performed, e.g., putting numerical weights on different PD types, for the greater scheme risk assessments.

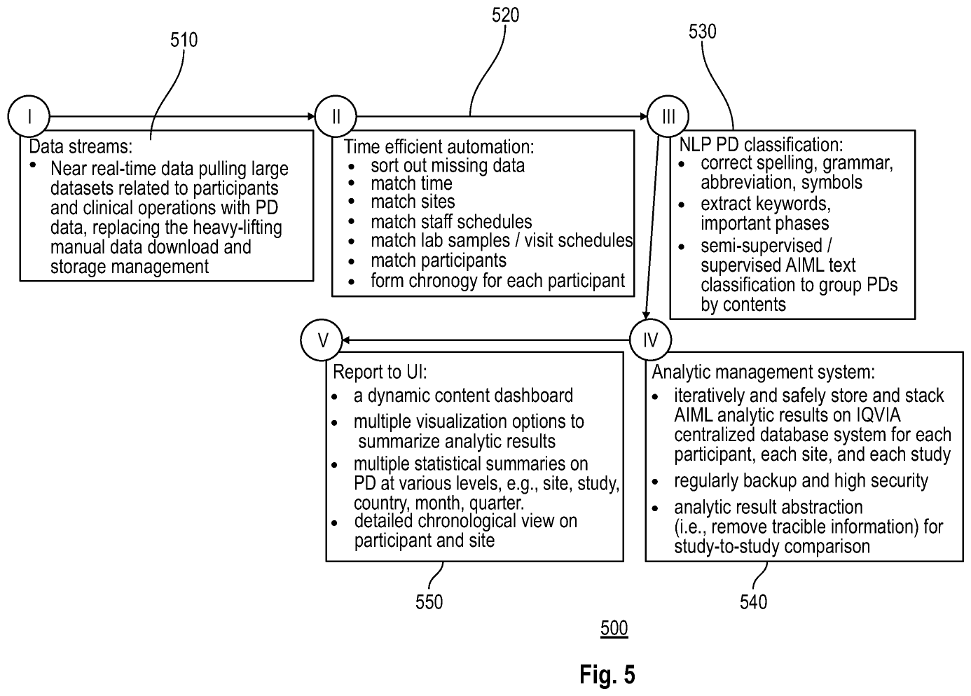

In FIG. 5, a flowchart 500 illustrating a process of the present disclosure is shown. At 510, real-time data of large datasets are pulled from a centralized database. The large datasets are related to participants and clinical operations with PD data. The data is downloaded from a centralized database by a cloud-computing server in place of any manual data download and storage management.

In FIG. 5, at 520, time efficient automation occurs. The cloud-computing server sorts out missing data and matches the time and sites with the PD levels. The cloud-computing server also matches staff schedules, lab samples, and visit schedules of the PDs. In addition, the cloud-computing server matches participants and forms a chronology for each participant relative to the PDs.

Referring to FIG. 5, at 530, the cloud-computing server performs natural language processing of the PDs to classify the PDs. The natural language processing includes correcting spelling, grammar, abbreviation and symbols of the dataset. In addition, the natural language processing includes extracting keywords and important phrases. Further, the natural language processing includes semi-supervised/supervised artificial intelligence/machine-learning text classification to group the PDs by content.

In FIG. 5 at 540, an analytic management system will iteratively and safely store and stack the artificial intelligence/machine-learning analytic results on the centralized database system for each participant, each site, and each study. The analytic management system will also regularly backup and have high security. In addition, the analytic management system will have analytic result abstraction, where traceable information is removed for study-to-study comparison.

In FIG. 5, at 550, the cloud-computing server reports to a user interface a dynamic content dashboard involving the pulled datasets with PDs. The cloud-computing server will provide multiple visualization options to summarize analytic results of the datasets. The cloud-computing server will also provide multiple statistical summaries on the PDs at various levels such as the site, study, country, month, and quarter. Further, the cloud-computing server will provide the user interface a detailed chronological view on each participate and each site.

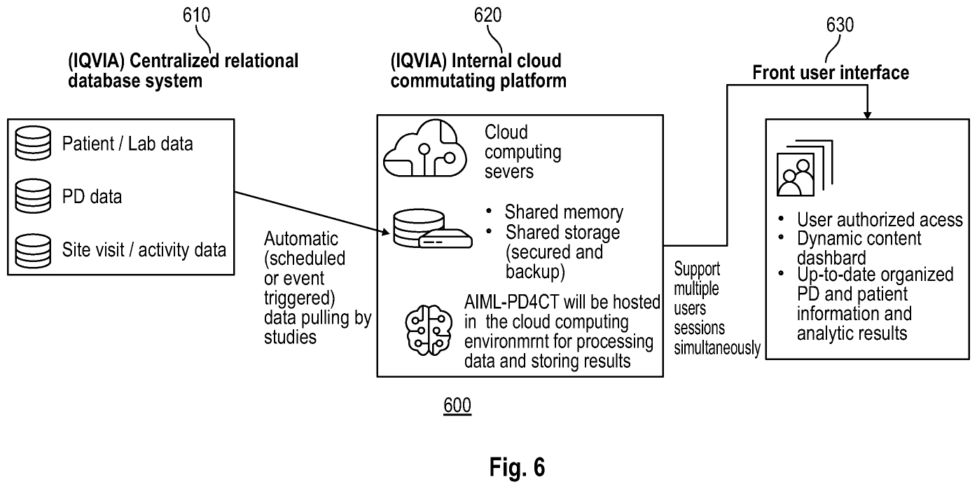

Referring to FIG. 6, the overall system 600 is illustrated. An IQVIA centralized relational database system (centralized database system) 610 is illustrated. The centralized database system 610 will include a large dataset of data. The dataset will include patient/lab data, PD data, and site visit/activity data. The IQVIA internal cloud-commutating platform (cloud commutating platform) 620 is also illustrated. The cloud-commutating platform 620 includes cloud computing servers, shared memory, shared storage (secured and backup). In addition, within the cloud-commuting platform 620, artificial intelligence machine learning (AIML)-(PD) 4CT will be hosted in the cloud-computing environment for processing data and storing results. At least one of the cloud-computing servers within the cloud-commuting platform 620 is configured with at least one processing unit. Moreover, at least one of the cloud-computing servers will put the dataset from the centralized database system 610. The cloud-computing server will put the patient/lab data, PD data, and site visit/activity data from the centralized database system in an efficient amount of time as opposed to a traditional download that would take more time.

In FIG. 6, once the cloud-computing server has pulled the data from the centralized database system 610, the cloud-computing server will perform AIML that involves content extraction to identify keywords, phrases, semi-supervised and supervised text to identify and group the PD data by content. The cloud-commuting platform 620 will report the results to a front user interface 630. The front user interface will include user authorized access and a dynamic content dashboard. In addition, the front user interface 630 will include up-to-date organized PD and patient information and analytic results.

Those skilled in the art will appreciate that the example embodiments are non-exhaustive and that embodiments other than that described here may be included without departing from the scope and spirit of the presently disclosed embodiments.

### Advantages

Overall, the system for monitoring PDs in clinical trials can enable the trends in the PDs to be identified and summarized. In addition, the PDs in the datasets can be compared with other studies with PDs to compare the PDs in the current dataset with historical studies with PDs. As such, the data of historical studies can be used as lessons learned to improve new protocols and avoid falling into the same traps with respect to PDs. Avoiding the same pitfalls as historical studies with regard to PDs would allow for safer and better quality study protocols, thereby resulting in higher study delivery success rates.

The cloud-computing server can pull the datasets from the centralized database. The cloud-computing server can perform the AI/ML on the PD data to extract content such as keywords, phrases, and semi-supervised and supervised text to group the PD data by content. As such, statistical summaries of the PDs can be provided. From the data, from the screening, to the number of patient visits, it can be determined where the trend in the PD is more likely.

Other advantages include being able to compare the PDs with PDs from other studies such as historical/baseline studies. From this comparison, it can be determined if the PDs have increased. In addition, it can also be determined if PDs are occurring at certain points that are the same as in the historical studies, or different than those of the historical studies.

Another advantage is there can be an early detection of potential risks in the current study with the PDs and also effective detection of widespread issues, such as identifying where the PDs are more likely to occur. In addition, expectations can be set of potential risks throughout the study can be set based on the historical data comparison. There can also be multiple statistical summaries on the PDs at various level s that include site, study, country, month, and quarter. Further, there can be a detailed chronological view on each participant and each site.

### Conclusion

All references, including granted patents and patent application publications, referred herein are incorporated herein by reference in their entirety.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Various aspects of the invention have been described above by way of illustration, and the specific embodiments disclosed are not intended to limit the invention to the particular forms disclosed. The particular implementation of the system provided thereof may vary depending upon the particular context or application. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

The corresponding structures, materials, acts, and equivalents of all means or step plus function elements in the claims below are intended to include any structure, material, or act for performing the function in combination with other claimed elements as specifically claimed.

1. A method comprising:
collecting a set of patient data from a centralized database, wherein the patient data is collected to identify protocol deviations within the patient data;
performing natural language processing and/or machine-learning by a cloud computing server on the protocol deviations to perform content extraction on the protocol deviations, wherein the content extraction is performed to extract keywords, phrases, and supervised text, wherein the extracted keywords, phrases, and supervised text are used to group the protocol deviations by content; and
reporting, to a user interface, multiple statistical summaries of the protocol deviations, wherein the multiple statistical summaries include a patient, site, study, and country.

2. The method of claim 1, further comprising:
performing time automation to identify missing data, match time and form a chronology for each patient identifier.

3. The method of claim 1, wherein a summary of analytical results of the protocol deviations are reported to the user interface.

4. The method of claim 1, wherein a chronological view for each patient identifier is provided to the user interface.

5. The method of claim 1, further comprising:
comparing the protocol deviations in one study with the protocol deviations found in other studies in the centralized database.

6. The method of claim 1, further comprising:
combining patient information and analytical results relative to the protocol deviations.

7. The method of claim 1, further comprising:
identifying protocol deviation trends and correlations along with patient visits among the patient data received from the centralized database.

8. A method comprising:
pulling datasets related to patient identifiers and clinical operations from a database system;
performing natural language processing of the pulled datasets related to the patient identifiers and clinical operations by a processing unit within a cloud computing server, wherein the natural language processing and other statistical/AIML base analytics are performed to identify trends and correlations among protocol deviations found within the datasets including patient visits, and classify and group the protocol deviations within the datasets, wherein the protocol deviation groups are obtained by keywords, phrases, and supervised text that are extracted by the natural language processing; and
storing analytical results of the protocol deviations from the natural language processing and the other statistical/AIML base analytics in an analytical management system.

9. The method of claim 8, wherein processing unit continuously receives updated patient and protocol deviation data from the database system for on-going studies.

10. The method of claim 8, wherein the processing unit uses artificial intelligence and machine-learning to extract the keywords, phrases, and supervised texts from the datasets.

11. The method of claim 8, further comprising:
making copies of the protocol deviation with corrected spelling and standardized names.

12. The method of claim 8, further comprising:
grouping the protocol deviations according to content extracted from the natural language processing.

13. The method of claim 8, further comprising:
matching lab samples and both patient and staff visit schedules from the datasets pulled from the database system.

14. The method of claim 8, further comprising:
reporting a chronological view for each patient identifier and site within the datasets to the user interface.

15. A system comprising:
a central database system with a set of non-identifiable patient data, wherein the patient data includes protocol deviations;
a cloud-computing server that pulls the set of patient data from the central database system, and performs national language processing and/or machine-learning on the protocol deviations to perform content extraction on the protocol deviations, wherein the content extraction is performed to extract keywords, phrases, and supervised text, wherein the extracted keywords, phrases, and supervised text are used to group the protocol deviations by content; and
a user interface receiving multiple statistical summaries of the protocol deviations from the cloud-computing server, wherein the multiple statistical summaries include a patient site, study, and country.

16. The system of claim 15, wherein the cloud-computing server reports a dynamic content dashboard to the user interface.

17. The system of claim 15, wherein patient identifiers with problems are identified from the protocol deviations.

18. The system of claim 15, wherein each patient identifier per each visit is statistically summarized.

19. The system of claim 15, wherein the protocol deviations from the patient data are compared to other data to enable untraceable patterns to be extracted.

20. The system of claim 15, wherein the clouding computing server reports to the user interface the protocol deviations of the patient data relative to countries, sites, and time involved.
